(19) United States
(12) Patent Application Publication
(10) Pub. No.: US 2005/0192600 A1
(43) Pub. Date: Sep. 1, 2005

(54) Inguinal hernia repair prosthetic

(76) Inventors: Enrico Nicolo, Jefferson Hill, PA (US); Massimo Mancusso, Napoli (IT)

Correspondence Address: BLYNN L. SHIDELER;THE BLK LAW GROUP, 3500 BROKKTREE ROAD, SUITE 200, WEXFORD, PA, 15090, US

(21) Appl. No.: 11/065,332

(22) Filed: Feb. 24, 2005

Related U.S. Application Data
(60) Provisional application No. 60/547,417, filed on Feb. 24, 2004.

(51) Int. Cl. A61B 17/08

(52) U.S. Cl. 606/151

(57) ABSTRACT

A suture-less, tension free, one-piece, double layer, inguinal hernia repair prosthetic is preferably formed of a single piece or sheet of repair fabric, such as polypropylene mesh, PTFE mesh, biologic material, or combinations thereof. The prosthetic sheet preferably includes a fold line dividing the sheet into two general halves. Each prosthetic half of the prosthetic sheet is preferably shaped generally to fit within the inguinal canal. Each prosthetic half of the sheet includes an opening in the inner portion of the sheet and a slit extending to the peripheral portion of the prosthetic half. The slits extend to generally opposite side of the prosthetic. Each opening is adapted to receive a patient's spermatic cord structure there through.

### RELATED APPLICATIONS

The present application claims the benefit of U.S. Patent Application Ser. No. 60/547,417 entitled “Inguinal Hernia Repair Prosthetic” filed Feb. 24, 2004.

### BACKGROUND

1. Field of the Invention

The present invention relates to a hernia repair prosthetic and, more particularly, to a suture-less, tension free, inguinal hernia repair prosthetic.

2. Discussion of the Related Art

Numerous surgically implantable hernia repair prosthetics have been proposed, such a polypropylene mesh patches for the repair of inguinal and other abdominal wall hernias. These prosthetics are intended for permanent placement within a patient's body space. These prosthetics are referred to as “tension free” surgical repairs of hernias and many have been developed using synthetic mesh materials to bridge and to patch hernia defects. These tension free repairs resulted in both a decrease in the recurrence rate as well as a decrease in the amount of a patient's post operative discomfort.

U.S. Pat. No. 5,593,441 discloses ventral hernia and/or chest wall reconstruction prosthesis that is a polypropylene mesh covered with an adhesion resistant barrier, such as a sheet of expanded PTFE. In the repair of ventral hernias and in chest wall reconstruction, the composite is positioned with the barrier relative to the region of potential adhesion, such as the abdominal viscera.

International Publication No. WO 97/35533 proposed a universal composite prosthesis in which one side of a layer of mesh material is completely covered with a layer of barrier material. The mesh material promotes biological tissue in-growth while the barrier material retards biological tissue adherence thereto.

U.S. Pat. No. 2,671,444 to Pease discloses a non-metallic mesh surgical insert for hernia repair. The non-metallic mesh surgical insert is preferably made from a polyethylene mesh.

Inguinal hernias are commonly repaired using a sheet of mesh fabric, such as polypropylene or PTFE (polytetraflouroethylene), which may be trimmed, as necessary, to match the particular size and shape of the inguinal floor. A slit is preformed or made by the surgeon from the lateral end of the mesh opposite the medial corner of the inguinal canal toward the medial end of the mesh to form a pair of lateral tails that are separated to receive the spermatic cord there between. The tails may then be overlapped to encircle the cord and reinforce the internal ring. A pre-shaped mesh may be provided with the slit and a keyhole at the end of the slit for receiving the cord therein.

U.S. Pat. No. 5,716,409 to Debbas discloses a reinforcement sheet for use in surgery that includes an opening to receive a spermatic cord and a passageway designed to allow the opening to fit around the spermatic cord.

U.S. Pat. No. 4,769,038 to Bendavid et al. discloses a plug for the repair of a femoral hernia. The plug or prosthesis has multiple layers or panels and fits around the spermatic cord.

PCT Application No. WO 96/14805 to Valenti discloses a double layer prosthesis that is applied to a patient with an inguinal hernia.

European Patent No. 0719527A1 to Sgro discloses dual layer prosthesis for an indirect inguinal hernia. The first layer is a solid sheet while the second layer has a center hole and a strip cut towards the center hole.

European Patent No. 0614650 to Rutkow discloses an implantable prosthesis that has a conical mesh plug having a pleated surface which conforms to the contours of the defect being repaired. Mesh filler material positioned on the plug stiffens the implant when it is compared to the defect.

U.S. published patent application No. 20010049538 is a set of plugs and hernia mesh pieces for a particular surgical technique. This technique involves exposing an approximately 12 centimeter by 4.5 centimeter box or opening in a patient. One of three kits can be used with this surgical technique with two kits for male patients and another kit for female patients. Each kit comprises a series of pre sterilized and pre tensioned mesh plugs that are cut to fit inside this box.

U.S. Pat. No. 6,174,320 to Kugel discloses a suture-less hernia repair patch having a slit for receiving a patient's chord structure when placing a patch in a patient for hernia repair.

Some of these earlier hernia repair prosthetics are complex. Several use a plug or a locating member to fit within the hernia defect itself. Also, many of these earlier techniques were designed specifically for use only in laparoscopic repair of hernias. Moreover, many of the prior inventions required suturing to the patient's body tissue. Although these medical advances are acknowledged for their usefulness and success, there remains a need or needs for more improvements in the surgical repair of hernias. In particular, a need exists for an improved implantable inguinal hernia repair prosthetic having an opening for receiving a patient's chord structure to facilitate the repair.

It is an object of the present invention to provide an improved method and prosthesis for the repair of inguinal hernias.

### SUMMARY OF THE INVENTION

The object of the invention is achieved with a suture-less, tension free, one-piece, double layer, inguinal hernia repair prosthetic according to the present invention. The repair prosthetic is preferably formed of a single piece or sheet of repair fabric, such as polypropylene mesh, PTFE mesh, biologic material, or combinations thereof. The prosthetic sheet preferably includes a fold line dividing the sheet into two general halves. Each prosthetic half of the prosthetic sheet is preferably shaped generally to fit within the inguinal canal. Each prosthetic half of the sheet includes an opening in the inner portion of the sheet and a slit extending to the peripheral portion of the prosthetic half. Each opening is adapted to receive a patient's spermatic cord structure there through, as generally known in the art.

In operation the single prosthetic sheet is folded along the fold line whereby the openings are coincident or aligned, thereby forming a one-piece two layer hernia repair prosthetic. It is preferred that the slits of prosthetic half extend to the periphery of the prosthetic on opposite sides of a medial plane bisecting the prosthetic halves. In a most preferred embodiment of the present invention the slits extend from the aligned opening in directions that are about 180 degrees apart.

Another aspect of the present invention provides that the two layer repair prosthetic is sized to repair the inguinal floor, with one of the layers (preferably the “upper” layer) being oversized to overlap the pubic spine to provide a reduction of reoccurrence. The non-oversized layer will preferably be sized to fit on the inguinal floor.

In one embodiment of the invention the prosthetic may be pre-folded by the manufacturer with the two layers attached to each other (other than the fold line) to hold the assembled prosthetic together. The attachment may be a weld, or other attachment technique, at the peripheral edges of the assembled prosthetic.

In another embodiment of the present invention the assembly (i.e. the folding and attachment, if any) may be left to the surgeon to provide greater flexibility in operation.

In a further embodiment of the present invention, the two layer prosthetic can be formed from two sheets attached together. This embodiment is similar in operation to the remaining embodiments except that the structure is formed from two separate sheets secured together (i.e. no fold line in the final assembled prosthetic).

The initial folded embodiment of the present invention has been described as folding in a “butterfly” motion. A further embodiment of this invention is a three layer, one piece structure in which there are two fold lines. This embodiment will fold the outer portions in on a central portion to form the three layer structure. A further embodiment is a two layer construction formed with two fold lines. This is similar to the three layer embodiment except that the outer portions combine together to form the second layer.

These and other advantages of the present invention will be clarified in the brief description of the preferred embodiments taken together with the attached figures in which like numerals represent like elements throughout.

### BRIEF DESCRIPTION OF THE DRAWINGS

FIG. 1 is a plan view of an un-assembled suture-less, tension free, one-piece, double layer, inguinal hernia repair prosthetic according to the present invention;

FIG. 2 is a plan view of an assembled suture-less, tension free, one-piece, double layer, inguinal hernia repair prosthetic according to the present invention;

FIG. 3 is a plan view of an assembled suture-less, tension free, one-piece, double layer, inguinal hernia repair prosthetic according to the present invention;

FIG. 4 is a plan view of a pre-assembled suture-less, tension free, one-piece, double layer, inguinal hernia repair prosthetic according to the present invention;

FIG. 5 is a plan view of a pre-assembled suture-less, tension free, double layer, inguinal hernia repair prosthetic according to the present invention;

FIG. 6 is a plan view of an un-assembled suture-less, tension free, one-piece, three layer, inguinal hernia repair prosthetic according to the present invention; and

FIG. 7 is a plan view of an assembled suture-less, tension free, one-piece, three layer, inguinal hernia repair prosthetic 10 according to the present invention.

### DESCRIPTION OF THE PREFERRED EMBODIMENTS

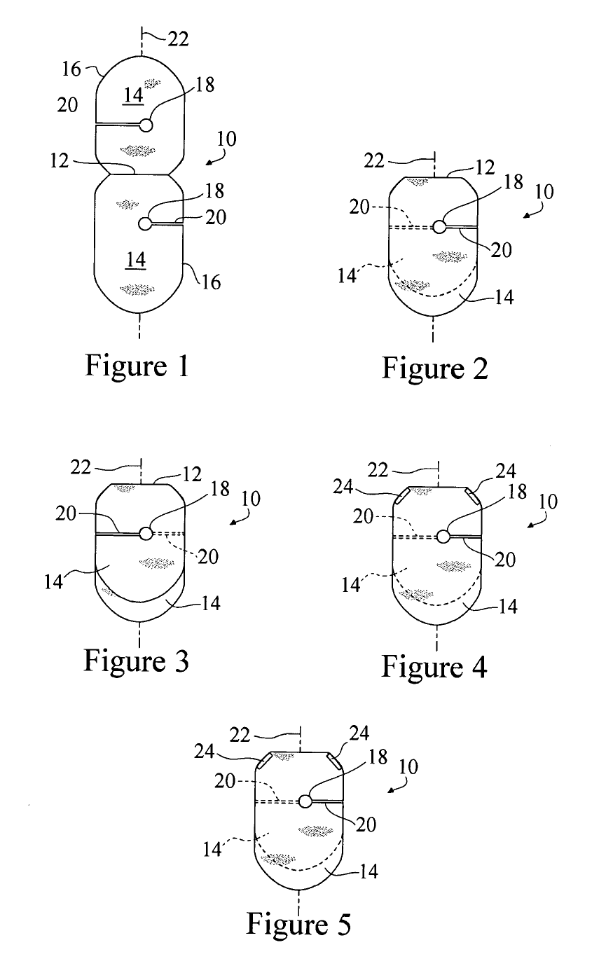

FIG. 1 is a plan view of an un-assembled suture-less, tension free, one-piece, double layer, inguinal hernia repair prosthetic 10 according to a first embodiment of the present invention. The repair prosthetic 10 is preferably formed of a single piece or sheet of repair fabric. The repair fabric may be formed of polypropylene mesh, or of PTFE mesh, or of ePTFE mesh, which are all known to be acceptable hernia repair prosthetic materials. Biologic materials can also be used, which may have certain advantages for use in the body. Finally, combinations of the aforementioned materials may also be used to form the single fabric. The present invention is intended to encompass any acceptable material for forming the fabric.

The sheet forming the prosthetic 10 includes a fold line 12 dividing the sheet into two prosthetic portions or halves 14. The fold line 12 may be formed as printed indicia indicative of where the operator may fold the sheet, or may be a perforation in the sheet or a thinner portion of the molded sheet to form the fold line 12. In the pre-assembled versions of the prosthetic 10 (i.e. where the prosthetic 10 is folded before the surgeon receives the product), the fold line 12 need not be demarcated at all since the assembly operation will form the fold line in the appropriate location. The prosthetic halves 14 need not be exactly equal in area as will be described below. Each prosthetic half 14 of the prosthetic sheet is preferably shaped generally to fit within the inguinal canal, and will therefore have a pre-shaped peripheral edge 16. The peripheral edge 16, within the meaning of this application is the edge of the sheet forming the prosthetic 10, and, therefore, does not include the fold line 12, even though in operation the fold line 12 can be found at the periphery of the prosthetic. The pre-shaping of the prosthetic 10 does not preclude the surgeon from trimming the prosthetic 10 as needed in operation. The pre-shaping is only intended to minimize the need for such trimming to expedite the operation.

Each prosthetic half 14 of the sheet includes an opening 18 in the inner portion of the prosthetic half 14 spaced from the peripheral edge 16 and the fold line 12. Each prosthetic half 14 further includes a slit 20 extending from the respective opening 18 to the peripheral edge 16 of the prosthetic half 14. Each opening 18 is adapted to receive a patient's spermatic cord structure there through, as generally known in the art. The opening 18 may be beaded or otherwise formed to minimize potential irritation to the cord structure extending there through.

In the operative state sheet of the prosthetic 10 is folded along the fold line 12 in a “butterfly” type action, whereby the openings 18 are coincident or aligned, thereby forming a one-piece two layer hernia repair prosthetic 10. As shown in FIGS. 2-5, the slits 20 of prosthetic halves 14 extend to the peripheral edge 16 of the prosthetic 10 on opposite sides of a medial plane 22 bisecting the prosthetic halves 14 along a longitudinal axis of the prosthetic. In other words the two slits 20 extend to opposite side edges of the prosthetic 10 as shown. It is preferred if the slits 20 extend from the aligned openings 18 in directions that are about 180 degrees apart, as generally shown.

As shown in the figures, the two layer repair prosthetic 10 is sized to repair the inguinal floor, with one of the prosthetic halves 14 or layers being oversized to overlap the pubic spine to provide a reduction of reoccurrence. The non-oversized prosthetic halve 14 is preferably be sized to fit on the inguinal floor. It is preferred in the oversized half 14 is on top of the undersized half 14 when it is laid on the inguinal floor such that the oversized half can easily overlay the pubic spine.

FIGS. 2 and 3 illustrate some of the flexibility of the prosthetic of the present invention when provided to the surgeon in an unfolded or unassembled condition. Through electing to fold the upper halve 14 of FIG. 1 either forward or backward, the surgeon can elect to have the slit 20 on the undersized halve extend to the desired side as shown in FIG. 2-3. This is purely up to the surgeon's preference and depends on whether he finds it easier to have the slit extend to the right or left side on the undersized half 14.

As will be understood by those of ordinary skill in the art, the surgeon will place the prosthetic 10 in position in the inguinal canal and introduce the cord structure of the patient into the opening 18 of the undersized halve 14 through the associated slit 20. After the cord structure is received in the first opening 18, the process is repeated for the second opening 18 using the other slit 20 on the opposite side of the prosthetic 10. The opposed slits 20 (opposed meaning that they extend in generally opposite directions) will prevent the cord structure from moving out of the aligned openings 18 of the prosthetic 10. The cord structure will serve to locate and position the prosthetic 10. The fabric or material of the prosthetic may be selected such that no other fixation is needed, thereby providing a suture-less prosthetic. The aforementioned known hernia mesh material such as polypropylene, PTFE and ePTFE are believed to be sufficient for the suture-less operation of the prosthetic 10 in inguinal hernia repair. It is important to note that the hernia repair prosthetic 10 does not exclude the use of sutures. The use of sutures to further secure the properly positioned prosthetic 10 is also acceptable as may be comfortable to the surgeon. As with all inguinal repair, care must be taken if sutures are used to avoid damaging sensitive structure in the inguinal canal.

Another option with the present invention is that the prosthetic 10 may be pre-folded by the manufacturer as shown in FIG. 4. The pre-assembly of the prosthetic 10 may further provide that the two layers formed by the respective halves 14 are attached to each other through welds 24 to hold the assembled prosthetic 10 together. The attachment may be welds 24 at the peripheral edges 16 of the assembled prosthetic 10 as shown, however, other attachment techniques and locations are possible. The limitation on the attachments, such as welds 24, is only that the respective slits 20 of each layer remain operational. In other words, the prosthetic halve 14 below the slit 20 must be freely moveable for easy placement of the prosthetic.

In a further embodiment of the present invention shown in FIG. 5, the two layer prosthetic 10 can be formed from two separate sheets attached together. Each sheet forms one of the halves 14 of the prosthetic 10. This embodiment is identical in operation to the remaining embodiments except that the prosthetic 10 is formed from two separate sheets secured together (i.e. no fold line 12 in the final assembled prosthetic 10). This embodiment is merely intended to show a manufacturing option for the prosthetic 10 of the present invention.

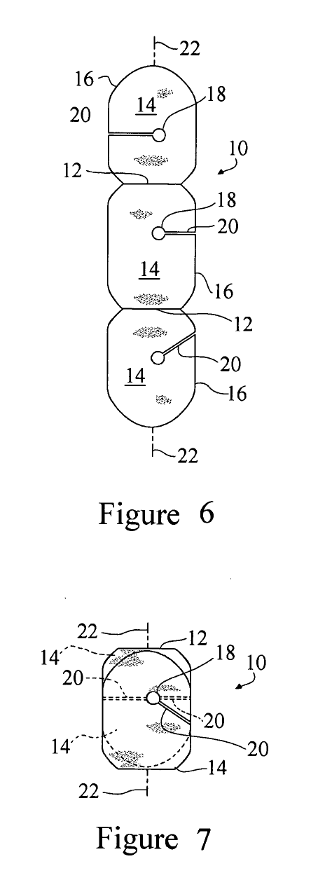

Another option with the present invention is that the prosthetic 10 may be more than two layers as shown in FIGS. 6 and 7. FIG. 6 is a plan view of an un-assembled suture-less, tension free, one-piece, three layer, inguinal hernia repair prosthetic 10 according to the present invention. FIG. 7 is a plan view of an assembled suture-less, tension free, one-piece, three layer, inguinal hernia repair prosthetic 10 according to the present invention. This is essentially the same as the prosthetic shown in FIG. 1, except that another portion 14 (which are no longer roughly “half” of the overall prosthetic 10, and fold line 12 is positioned on the prosthetic 10. Folding the two outer portions 14 along the fold line will create a one piece, three layer prosthetic in essentially the same manner as the two layer version was formed above. The only addition is repeating the folding process for the remaining portion 14. The openings 18 are all aligned in the operative position, with the respective slits 20 being un-aligned or offset. Preferably the slit 20 in the middle layer (formed by one portion 14) will extend to the opposite side of the prosthetic 10 from the remaining two outer layers (formed by the remaining two portions 14). With this construction the slits 20 of adjacent layers will extend in generally opposite directions. The two slits 20 that extend to the same side of the prosthetic when assembled should also be offset from each other to provide some locking or holding between these two layers in the event the prosthetic is assembled with these two layers adjacent to each other.

The above described embodiments are intended to be illustrative of the present invention and not restrictive thereof. It will be apparent to those of ordinary skill in the art that various modification and additions may be made to the above described invention without departing from the spirit and scope thereof. The scope of the present invention is intended to encompass all such modifications and additions and will be defined by the appended claims and equivalents thereto.

1. A one piece, multiple layer inguinal hernia repair prosthetic comprising a single sheet of repair fabric having:
A) at least one fold line dividing the sheet into at least two prosthetic portions, wherein the prosthetic portions overlap when the repair fabric is folded along the fold line to form a multiple layer prosthetic;
B) an opening through the repair fabric formed in each prosthetic portion and spaced from the peripheral edge of the repair fabric, wherein each opening is adapted to receive the spermatic cord there through, and wherein the openings are adapted to be substantially aligned when the repair fabric is folded along the fold line to overlap sufficiently in plan view to allow the cord to pass there through; and
C) an access slit extending from each opening in each prosthetic portion to the peripheral edge of the repair fabric, wherein each access slit allows the spermatic cord to be placed through the associated opening, and wherein the access slit in each prosthetic portion is not aligned with the access slit of an adjacent layer when the prosthetic portions are folded along the fold lines.

2. The one piece, multiple layer inguinal hernia repair prosthetic of claim 1 wherein the repair fabric is formed of one of polypropylene mesh, PTFE mesh, ePTFE mesh, and biologic material.

3. The one piece, multiple layer inguinal hernia repair prosthetic of claim 1 wherein only a single fold line is provided dividing the sheet into two prosthetic portions of substantially half of the prosthetic.

4. The one piece, multiple layer inguinal hernia repair prosthetic of claim 1 wherein prosthetic is provided to the operator un-folded and wherein the fold line is formed as printed indicia indicative of where the operator is to fold the sheet

5. The one piece, multiple layer inguinal hernia repair prosthetic of claim 1 wherein each opening is beaded to minimize potential irritation to the cord structure extending there through.

6. The one piece, multiple layer inguinal hernia repair prosthetic of claim 1 wherein exactly two prosthetic portions are provided and the slits of the two prosthetic portions extend to the peripheral edge of the prosthetic on opposite sides of a medial plane bisecting the prosthetic portions along a longitudinal axis of the prosthetic.

7. The one piece, multiple layer inguinal hernia repair prosthetic of claim 1 wherein the repair prosthetic is pre-shaped to repair the inguinal floor.

8. The one piece, multiple layer inguinal hernia repair prosthetic of claim 1 wherein one of the prosthetic portions is pre-shaped to repair the inguinal floor and oversized to overlap the pubic spine to provide a reduction of reoccurrence.

9. The one piece, multiple layer inguinal hernia repair prosthetic of claim 1 wherein one of the prosthetic portions is pre-shaped to repair the inguinal floor and sized to fit on the inguinal floor.

10. The one piece, multiple layer inguinal hernia repair prosthetic of claim 1 wherein the prosthetic is pre-folded by the manufacturer and further includes at least one attachment between the layers to hold the assembled prosthetic together.

11. The one piece, multiple layer inguinal hernia repair prosthetic of claim 1 wherein at least two fold lines are provided dividing the sheet into at least three prosthetic portions.

12. The one piece, multiple layer inguinal hernia repair prosthetic of claim 1 wherein a two fold lines are provided dividing the sheet into three prosthetic portions and wherein the slits of adjacent layers extend in generally opposite directions.

13. A multiple layer inguinal hernia repair prosthetic comprising:
A) at least two prosthetic portions, wherein the prosthetic portions overlap to form a multiple layer prosthetic;
B) an opening through the repair fabric formed in each prosthetic portion and spaced from the peripheral edge of the repair fabric, wherein each opening is adapted to receive the spermatic cord there through, and wherein the openings are adapted to be substantially aligned when the repair fabric is folded along the fold line to overlap sufficiently in plan view to allow the cord to pass there through; and
C) an access slit extending from each opening in each prosthetic portion to the peripheral edge of the repair fabric, wherein each access slit allows the spermatic cord to be placed through the associated opening, and wherein the access slit in each prosthetic portion is not aligned with the access slit of an adjacent layer in the prosthetic; and
D) at least one attachment between the layers to hold the assembled prosthetic together.

14. The multiple layer inguinal hernia repair prosthetic of claim 13 wherein the repair fabric is formed of one of polypropylene mesh, PTFE mesh, ePTFE mesh, and biologic material.

15. The multiple layer inguinal hernia repair prosthetic of claim 13 wherein each opening is beaded to minimize potential irritation to the cord structure extending there through.

16. The multiple layer inguinal hernia repair prosthetic of claim 13 wherein exactly two prosthetic portions are provided and the slits of the two prosthetic portions extend to the peripheral edge of the prosthetic on opposite sides of a medial plane bisecting the prosthetic portions along a longitudinal axis of the prosthetic.

17. The multiple layer inguinal hernia repair prosthetic of claim 13 wherein the repair prosthetic is pre-shaped to repair the inguinal floor, wherein one of the prosthetic portions is pre-shaped to repair the inguinal floor and oversized to overlap the pubic spine to provide a reduction of reoccurrence, and wherein one of the prosthetic portions is pre-shaped to repair the inguinal floor and sized to fit on the inguinal floor.

18. The multiple layer inguinal hernia repair prosthetic of claim 13 wherein the prosthetic includes at least three prosthetic portions.

19. The multiple layer inguinal hernia repair prosthetic of claim 13 wherein exactly three prosthetic portions are provided and wherein the slits of adjacent layers extend in generally opposite directions.

20. A suture-less, one piece, two layer inguinal hernia repair prosthetic comprising a single sheet of repair fabric mesh having:
A) a fold line dividing the sheet into two pre-shaped prosthetic halves, wherein the prosthetic halves overlap when the repair fabric is folded along the fold line to form the two layer prosthetic;
B) an opening through the repair fabric formed in each prosthetic half and spaced from the peripheral edge of the repair fabric, wherein each opening is adapted to receive the spermatic cord there through, and wherein the openings are adapted to be substantially aligned when the repair fabric is folded along the fold line to overlap sufficiently in plan view to allow the cord to pass there through; and
C) an access slit extending from each opening in each prosthetic half to the peripheral edge of the repair fabric, wherein each access slit allows the spermatic cord to be placed through the associated opening, and wherein the access slit in each prosthetic portion is not aligned with the access slit of an adjacent layer when the prosthetic portions are folded along the fold lines, and wherein the slits of the two prosthetic halves extend to the peripheral edge of the prosthetic on opposite sides of a medial plane bisecting the prosthetic halves along a longitudinal axis of the prosthetic.
